(19) United States
(12) Patent Application Publication
(10) Pub. No.: US 2021/0186907 A1
(43) Pub. Date: Jun. 24, 2021

(54) GHB DOSING

(71) Applicant: JAZZ PHARMACEUTICALS IRELAND LIMITED, Dublin (IE)

(72) Inventor: Franck SKOBIERANDA, Flourtown, PA (US)

(21) Appl. No.: 17/130,769

(22) Filed: Dec. 22, 2020

Related U.S. Application Data
(60) Provisional application No. 63/052,676, filed on Jul. 16, 2020.
(60) Provisional application No. 63/000,547, filed on Mar. 27, 2020.
(60) Provisional application No. 62/993,372, filed on Mar. 23, 2020.
(60) Provisional application No. 62/953,288, filed on Dec. 24, 2019.

(51) Int. Cl. A61K 31/19; A61K 9/00; A61P 25/00

(52) U.S. Cl. CPC A61K 31/19; A61P 25/00; A61K 9/0053

(57) ABSTRACT

Provided herein are methods of administering GHB formulations for the treatment of narcolepsy and other conditions.

### CROSS-REFERENCE TO RELATED APPLICATIONS

This application claims the benefit of priority to U.S. Application Ser. No. 62/953,288, filed Dec. 24, 2019, U.S. Application Ser. No. 62/993,372, filed Mar. 23, 2020, U.S. Application Ser. No. 63/000,547, filed Mar. 27, 2020, and U.S. Application Ser. No. 63/052,676, filed Jul. 16, 2020, the contents of each of which are hereby incorporated by reference in their entireties for all purposes.

### BACKGROUND

Gamma-hydroxybutyrate (GHB), also known as “oxybate,” is an endogenous compound with hypnotic properties that is found in many human body tissues. GHB is present, for example, in the mammalian brain and other tissues. In the brain, the highest GHB concentration is found in the hypothalamus and basal ganglia and GHB is postulated to function as a neurotransmitter (Snead and Morley, 1981, Brain Res. 227(4): 579-89). The neuropharmacologic effects of GHB include increases in brain acetylcholine, increases in brain dopamine, inhibition of GABA-ketoglutarate transaminase and depression of glucose utilization but not oxygen consumption in the brain. GHB treatment substantially reduces the signs and symptoms of narcolepsy, i.e., daytime sleepiness, cataplexy, sleep paralysis, and hypnagogic hallucinations. In addition, GHB increases total sleep time and REM sleep, and it decreases REM latency, reduces sleep apnea, and improves general anesthesia (e.g., U.S. Pat. Nos. 6,472,431; 6,780,889; 7,262,219; 7,851,506; 8,263,650; and 8,324,275, the disclosure of each of which is incorporated by reference in its entirety for all purposes).

Sodium oxybate (Na.GHB), commercially sold as Xyrem®, is approved for the treatment of excessive daytime sleepiness and cataplexy in patients with narcolepsy. Na.GHB has also been reported to be effective for relieving pain and improving function in patients with fibromyalgia syndrome (See Scharf et al., 2003, J. Rheumatol. 30: 1070; Russell et al., 2009, Arthritis. Rheum. 60: 299), in treating alcohol addiction and alcohol withdrawal syndrome (See Keating, G M, 2014, January; 34(1):63-80), in alleviating excessive daytime sleepiness and fatigue in patients with Parkinson's disease, improving myoclonus and essential tremor, and reducing tardive dyskinesia and bipolar disorder (See Ondo et al., 2008, Arch. Neural. 65: 1337; Frucht et al., 2005, Neurology 65: 1967; Berner, 2008, J. Clin. Psychiatry 69: 862).

Xyrem®, for use with patients with narcolepsy, is a chronically used product that requires high dose strengths of the drug. The amount of sodium intake from the drug significantly increases the dietary sodium intake for patients, which is undesirable for all patients, and especially those with cardiometabolic risk, such as patients with heart failure, hypertension, or impaired renal function. Thus, there is a need in the art for oxybate compositions and treatment methods that provide reduced patient sodium intake compared to Xyrem®.

### SUMMARY

In one aspect, the present disclosure provides methods of substituting, exchanging, changing or switching a mixed salt oxybate composition for a sodium oxybate composition in a patient treated with sodium oxybate (such as a patient treated for cataplexy, excessive daytime sleepiness in patients with narcolepsy, or idiopathic hypersomnia), wherein the amount of the sodium oxybate and mixed salt oxybate are the same on an oxybate dosing strength basis.

In some embodiments, the mixed salt oxybate comprises about 8% mol. equiv. of sodium oxybate, about 23% mol. equiv. of potassium oxybate, about 21% mol. equiv. of magnesium oxybate and about 48% mol. equiv. of calcium oxybate.

In one aspect, the present disclosure provides for switching a patient who is administered sodium oxybate for the treatment of cataplexy or excessive daytime sleepiness in patients with narcolepsy to a mixed salt oxybate composition, the method comprising:
administering a therapeutically effective amount a mixed salt oxybate to the patient, wherein the amount of sodium oxybate and the mixed salt oxybate are the same on an oxybate dosing strength basis.

In one aspect, the present disclosure provides for treating cataplexy or excessive daytime sleepiness in patients with narcolepsy, the method comprising:
switching the dose of a patient who is administered sodium oxybate to a mixed salt oxybate, wherein the switching comprises administering a therapeutically effective amount of the mixed salt oxybate to the patient and wherein the amount of sodium oxybate and mixed salt oxybate are the same on an oxybate dosing strength basis.

In some embodiments, about 0.5 g-9 g of the mixed salt oxybate is administered per day. In some embodiments, about 0.5 g of the mixed salt oxybate is administered per day. In some embodiments, about 0.25 g of the mixed salt oxybate is administered twice per day. In some embodiments, about 1.0 g of the mixed salt oxybate is administered per day. In some embodiments, about 0.5 g of the mixed salt oxybate is administered twice per day. In some embodiments, about 1.5 g of the mixed salt oxybate is administered per day. In some embodiments, about 0.75 g of the mixed salt oxybate is administered twice per day. In some embodiments, about 2.0 g of the mixed salt oxybate is administered per day. In some embodiments, about 1.0 g of the mixed salt oxybate is administered twice per day. In some embodiments, about 2.5 g of the mixed salt oxybate is administered per day. In some embodiments, about 1.25 g of the mixed salt oxybate is administered twice per day. In some embodiments, about 3.0 g of the mixed salt oxybate is administered per day. In some embodiments, about 1.5 g of the mixed salt oxybate is administered twice per day. In some embodiments, about 3.5 g of the mixed salt oxybate is administered per day. In some embodiments, about 1.75 g of the mixed salt oxybate is administered twice per day. In some embodiments, about 4.0 g of the mixed salt oxybate is administered per day. In some embodiments, about 2.0 g of the mixed salt oxybate is administered twice per day. In some embodiments, about 4.5 g of the mixed salt oxybate is administered per day. In some embodiments, about 2.25 g of the mixed salt oxybate is administered twice per day. In some embodiments, about 6 g of the mixed salt oxybate is administered per day. In some embodiments, about 3 g of the mixed salt oxybate is administered twice per day. In some embodiments, about 7.5 g of the mixed salt oxybate is administered per day. In some embodiments, about 3.75 g of the mixed salt oxybate is administered twice per day. In some embodiments, about 9 g of the mixed salt oxybate is administered per day. In some embodiments, about 4.5 g of the mixed salt oxybate is administered twice per day.

In some embodiments, the mixed salt oxybate is administered at bedtime. In some embodiments, the mixed salt oxybate is administered at bedtime and about 2.5 h-4 h after the bedtime administration.

In some embodiments, mixed salt oxybate is in a liquid. In some embodiments, the concentration of the mixed salt in the liquid is about 0.5 g/mL.

In some embodiments, the patient is treated for cataplexy. In some embodiments, the patient is treated for excessive daytime sleepiness in patients with narcolepsy. In some embodiments, the patient is treated for idiopathic hypersomnia.

### BRIEF DESCRIPTION OF THE FIGURES

FIG. 1 shows mean plasma oxybate concentration-time profiles for Xyrem and JZP-258 under fasted and fed conditions from patients in Example 1, Study 1.

FIG. 2 shows mean plasma oxybate concentration-time profiles for Xyrem and JZP-258 under fasted and fed conditions from patients in Example 1, Study 2.

FIG. 3 shows the disposition of subjects in the study of Example 2 evaluating the efficacy of JZP-258. Patients entered the open-label optimized treatment and titration period, where the dose of JZP-258 could be adjusted if needed to provide a stable, tolerable, and effective dose.

### DEFINITIONS

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The terms “administer,” “administering” or “administration” as used herein refer to directly administering a compound or pharmaceutically acceptable salt of the compound or a composition or formulation comprising the compound or pharmaceutically acceptable salt of the compound to a patient.

As used herein, the term “gamma-hydroxybutyrate” (GHB) or “oxybate” refers to the negatively charged or anionic form (conjugate base) of gamma-hydroxybutyric acid. GHB has the following structural formula:

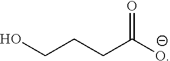

As used herein, the term “gamma-hydroxybutyric acid” (GBA) refers to the protonated form (conjugate acid) of gamma-hydroxybutyrate. GBA has the following structural formula:

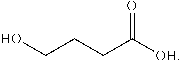

Salt forms of GHB are disclosed in U.S. Pat. Nos. 8,591,922; 8,901,173; 9,132,107; 9,555,017; and 10,195,168, which are hereby incorporated by reference in their entireties for all purposes.

The terms “effective amount” and “therapeutically effective amount” are used interchangeably in this disclosure and refer to an amount of a compound, or a salt thereof, that, when administered to a patient, is capable of performing the intended result. For example, an effective amount of a mixed salt oxybate is that amount which is required to reduce cataplexy in a patient. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The term “equivalent” when comparing Na.GHB and mixed salts forms contains the same amount of GHB within about 5% (by weight %). In preferred embodiments, a liquid formulation of a mixed salt is equivalent to the Na.GHB-containing liquid formulation Xyrem (which contains 0.409 g/mL of GHB).

In preferred embodiments, a liquid formulation of a mixed salt contains 0.234 g/mL of calcium oxybate, 0.130 g/mL of potassium oxybate, 0.096 g/mL of magnesium oxybate, and 0.040 g/mL of sodium oxybate (which contains 0.413 g/mL of GHB).

As used herein, the term “patient” refers to a mammal, particularly a human.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “carrier” encompasses solvents, dispersion media, coatings, antibacterial and antifungal agents, isotonic and absorption delaying agents and the like. The use of carriers for active pharmaceutical ingredients is well known in the art. Insofar as any conventional media or agent is incompatible with the active ingredient, its use in the therapeutic compositions is not appropriate.

The term “therapeutic effect” as used herein refers to a desired or beneficial effect provided by the method and/or the composition. For example, the method for treating cataplexy provides a therapeutic effect when the method reduces cataplexy.

The term “treating” as used herein with regard to a patient, refers to improving at least one symptom of the patient's disorder. Treating can be curing, improving, or at least partially ameliorating a disorder.

The terms “substitute”, “switch”, “change” and “exchange” are used interchangeably in the context of the present disclosure. The methods of the present disclosure may also be expressed in terms of “transitioning from” sodium oxybate to a mixed salt oxybate.

The term “salt” or “salts,” as used herein, refers to a compound formed by the interaction of an acid and a base, the hydrogen atoms of the acid being replaced by the positive ion or cation of the base. Pharmaceutically acceptable salts, include inorganic acids such as, for example, hydrochloric or phosphoric acids, or such organic acids as malic, acetic, oxalic, tartaric, mandelic, and the like. Salts formed can also be derived from inorganic bases such as, for example, sodium, potassium, silicates, ammonium, calcium, or ferric hydroxides, and such organic bases as isopropylamine, trimethylamine, histidine, procaine and the like. In certain preferred embodiments, the salt is formed from an inorganic base that is a metal, for example, an alkali metal, such as lithium, potassium, sodium, or the like, an alkaline earth metal, such as magnesium, calcium, barium, or the like, or aluminum or zinc. Other salts may comprise ammonium. Alkali metals, such as lithium, potassium, sodium, and the like, may be used, preferably with an acid to form a pH adjusting agent. Examples of pharmaceutically acceptable base addition salts include those derived from inorganic bases like sodium hydroxide, potassium hydroxide, magnesium hydroxide, calcium hydroxide, or ammonium hydroxide, and the like (See, e.g., Berge et al., 1977, J. Pharm. Sci. 66: 1).

As used herein, the terms “salt of GHB” or “salts of GHB,” as used herein, refer to a compound formed by the interaction of gamma-hydroxybutyric acid (the conjugate acid of GHB) with a base, for example, NaOH, KOH, Mg(OH)₂, and Ca(OH)₂, and the like, the hydrogen atoms of the acid being replaced by the positive ion or cation of the base. Such salts may include, for example, sodium oxybate (“Na.GHB”), potassium oxybate (“K.GHB”), magnesium oxybate (“Mg.(GHB)₂”), and calcium oxybate (“Ca.(GHB)₂”), and the like. It will be understood by those skilled in the art that such salts may be in solid form, or such salts may be in partially or fully solvated form, for example, as when dissolved in an aqueous medium. It will be further understood by those skilled in the art, that, depending on the solubility of the salt in the aqueous medium, that the salt may be present in the aqueous medium as solvated cation(s) and anion(s), or as a precipitated solid.

The term “oxybate dosing strength” refers to the amount of GHB in a particular dose (e.g., each mL of Xyrem contains 0.5 g of sodium oxybate, which is equivalent to a 0.409 g/mL oxybate dosing strength). Although throughout the present disclosure, the oxybate dosing strength in a composition is generally expressed in terms of the amount of oxybate present in a composition, the present disclosure contemplates embodiments where the oxybate dosing strength is expressed in the Equivalent Concentration of GBA that is contained in the dose.

The Equivalent Concentration of GBA in a compositions may be calculated by the following formula:

$$Equivalent Concentration of GBA = \\ \frac{Concentration of GHB in ( g / mL ) \times 104.1 ( Formula Weight of GBA , \frac{g}{mol} )}{103.1 ( Formula Weight of GHB ( \frac{g}{mol} )}$$

Thus, each mL of Xyrem contains 0.5 g of sodium oxybate, which is equivalent to an Equivalent Concentration of GBA of 0.413 g/mL.

The term “JZP-258” as used herein refers to a solution containing the mixed salt oxybate comprising about 8% sodium oxybate, about 23% potassium oxybate, about 21% magnesium oxybate and about 48% calcium oxybate (% mol. equiv. of GHB) and having a GHB concentration of 0.409 g/mL (or, expressed another way, an Equivalent Concentration of GBA of 0.413 g/mL). The following table describes the % mol. equiv., wt/vol %, and absolute amount of sodium oxybate, potassium oxybate, magnesium oxybate and calcium oxybate in representative doses of JZP-258.

| | | | Amount in 1 | Amount in 9 |
|---|---|---|---|---|
| | | | mL JZP-258 | mL JZP-258 |
| | % mol equivalent | wt/vol % | solution | solution |
| Na.GHB | 8 | 8 | 40 mg | 720 mg |
| K. GHB | 23 | 26 | 130 mg | 2,340 mg |
| Mg.(GHB)₂ | 21 | 19.2 | 96 mg | 1,728 mg |
| Ca.(GHB)₂ | 48 | 46.8 | 234 mg | 4,212 mg |

The term “mixed salts” or “mixed salt oxybate,” as used herein, refers to salts of GHB where two, three, four or more different cations are present in combination with each other in a composition. Such mixtures of salts may include, for example, salts selected from the group consisting of Na.GHB, K.GHB, Mg.(GHB)₂, and Ca.(GHB)₂. Mixed salt oxybates are described in U.S. Pat. Nos. 8,591,922; 8,901,173; 9,132,107; 9,555,017; and 10,195,168, the contents of which is hereby incorporated by reference it entirety for all purposes.

The term “wt/wt %,” as used herein, refers to the normalized weight percent of a particular salt in a salt mixture.

The term “wt/wt % ratio,” as used herein, refers to the ratio of wt/wt % values in a mixture of salt. For example, where the salts Na.GHB, K.GHB, Mg.(GHB)₂, and Ca.(GHB)₂ are present in a wt/wt %'s of 8%, 25.5%, 19.5% and 47%, respectively, the wt/wt % ratio of Na.GHB, K.GHB, Mg.(GHB)₂, and Ca.(GHB)₂ in the mixture is 8:25.5:19.5:47.

The term “wt/vol %,” as used herein, refers to the normalized weight percent of a particular salt in a particular volume of solution.

The term, “formulation,” as used herein, refers to a stable and pharmaceutically acceptable preparation of a pharmaceutical composition disclosed herein.

The term, “liquid formulation,” as used herein, refers to a water-based formulation, in particular, a formulation that is an aqueous solution.

### DETAILED DESCRIPTION

Sodium oxybate (Na.GHB), commercially sold as Xyrem®, is approved for the treatment of cataplexy or excessive daytime sleepiness in patients 7 years of age or older with narcolepsy. Administration of the approved daily dose of Xyrem® (6-9 grams per night administered orally) results in the adult patient ingesting from 1100-1638 mg of sodium daily. The American Heart Association has recommended a daily sodium intake of less than 2300 mg and an “ideal” daily intake of <1500 mg (AHA 2017; Whelton 2012), and a recent report from The National Academies of Science, Engineering, and Medicine (2019) advises adults to “reduce intake if above 2300 mg/day” based on strong causal evidence of cardiovascular disease risk above this level. Thus, Xyrem® administration provides a sodium intake that makes up a substantial amount of the recommended daily intake goals, which renders adherence to daily sodium intake goals challenging since—even without the consideration of Xyrem—the average daily sodium intake for Americans≥2 years of age is >3400 mg (US Department of Agriculture, Agricultural Research Service. Nutrient intakes from food: mean amounts consumed per individual, by gender and age, in the United States, 2009-2010. In: What We Eat in America, NHANES 2009-2010. Washington, D.C.: US Department of Agriculture, Agricultural Research Service; 2012.).

JZP-258 (a preferred embodiment of the present disclosure) was developed to provide the same treatment benefits as Xyrem with substantially less sodium, so that patients with the lifelong disease of narcolepsy could be more able to achieve daily sodium intake goals for optimum health.

JZP-258 is a mixed salt oxybate that contains calcium oxybate, magnesium oxybate, potassium oxybate, and sodium oxybate, and it provides 87-131 mg of sodium when administered in the dose range of 6-9 grams nightly. This amount is 92% less sodium than that provided by Xyrem® administration of an equivalent dose. Though important for every person, daily sodium intake goals are a vital consideration for all patients with the lifelong disease of narcolepsy, given the increased presence of multiple cardiovascular comorbidities, including hypertension, congestive heart failure, and myocardial infarction (Jennum P, et al. Comorbidity and mortality of narcolepsy: a controlled retro- and prospective national study. Sleep. 2013 Jun. 1; 36(6):835-40.; Ohayon M M. Narcolepsy is complicated by high medical and psychiatric comorbidities: a comparison with the general population. Sleep Med. 2013 June; 14(6):488-92.; and Black J, et al. Medical comorbidity in narcolepsy: findings from the Burden of Narcolepsy Disease (BOND) study. Sleep Med. 2017 May; 33:13-18.). Thus, patients on Xyrem® therapy could benefit by switching from sodium oxybate to a mixed salt oxybate that provides the needed therapeutic benefit but provides less dietary sodium when administered.

However, switching a patient from one drug therapy to another is challenging as it is not predictable what the efficacious dose of the new therapy will be or even whether the new therapy will be efficacious at all. The present disclosure relates to unexpected findings during the development of one embodiment of the present disclosure, JZP-258.

During the development of JZP-258, it was found that although the pharmacokinetic characteristics of JZP-258 and Xyrem® were similar, bioequivalence was not established since the JZP-258 exhibited: a) an approximately 20% lower Cmax compared with Xyrem® under fasted conditions, b) a longer time to maximum concentration compared with Xyrem® under fasted conditions, and c) a lesser food effect compared with Xyrem® (Example 1).

Because bioequivalence was not demonstrated, a Phase 3 study was conducted to support the safety and efficacy of JZP-258 (Example 2). The study involved four patient groups with narcolepsy at study entry:
Group 1: Patients taking Xyrem® prior to study;
Group 2: Patients taking Xyrem® with other drugs aimed to treat the cataplexy symptom of narcolepsy (“other anticataplectics”) prior to study;
Group 3: Patients taking other anticataplectics prior to study; and
Group 4: Patients not taking Xyrem® or other anticataplectics prior to study. (“Xyrem®—naïve”).

Group 1 and 2 subjects were switched from Xyrem® to JZP-258 (gram for gram of GHB), administered JZP-258 dose for a minimum of 2 weeks, and then the dose was titrated during the subsequent 8 weeks to provide a stable, tolerable, and effective dose. Because it had been established that that Xyrem® and JZP-258 were not bioequivalent, it was expected that the dose of JZP-258 would need to be significantly adjusted during the titration period. However, this was not observed and, instead, it was unexpectedly found that most patients who switched from Xyrem® to JZP-258 (69.5%) remained on the same dose strength and, in most patients for whom a dose adjustment was made, the change was moderate (i.e., within 1.5 grams, i.e., one incremental dose change).

Thus, the present disclosure provides methods of switching a patient from sodium oxybate to a mixed salt oxybate, where the amount of sodium oxybate and mixed salt oxybate are the same on an oxybate dosing strength basis (i.e., the amount of GHB administered to the patient in the sodium oxybate administration and the amount of GHB administered to the patient in the mixed salt oxybate administration are the same).

The following patents, publications and application are related to the present disclosure and are hereby incorporated by reference in their entireties for all purposes: U.S. Pat. Nos. 6,472,431; 6,780,889; 7,262,219; 8,263,650; 8,461,203; 8,859,619; 9,539,330; 7,851,506; 8,324,275; 8,952,062; 8,731,963; 8,772,306; 8,952,029; 9,050,302; 9,486,426; 10,213,400; 8,591,922; 8,901,173; 9,132,107; 9,555,017; 10,195,168; 8,778,301; 9,801,852; 8,771,735; 8,778,398; 9,795,567; U.S. Patent Publication Nos. US 2018/0042855, and U.S. application Ser. No. 16/688,797, 62/769,380 and 62/769,382.

### Mixed Salt Oxybate

In some embodiments, the methods of the present disclosure comprise administering a mixed salt oxybate to a patient in need thereof.

In some embodiments, the mixed salt oxybate comprises gamma-hydroxybutyrate (GHB) and three or four or more pharmaceutically acceptable cations of an alkali metal or an alkaline earth metal.

In some embodiments, the mixed salt oxybate comprises GHB and more than one pharmaceutically acceptable cations of an alkali metal or an alkaline earth metal.

In some embodiments, the mixed salt oxybate comprises GHB and two, three, or four cations selected from the group consisting of Na⁺, K⁺, Mg⁺², and Ca⁺². In some embodiments, mixed salt oxybate comprises GHB and all three cations selected from the group consisting of K⁺, Mg⁺², and Ca⁺². In some embodiments, the mixed salt oxybate does not contain Na⁺, or comprises less of, Na⁺.

In some embodiments, the mixed salt oxybate comprises two, three, or four salts selected from the group consisting of a sodium salt of hydroxybutyrate (Na.GHB), a potassium salt of gamma-hydroxybutyrate (K.GHB), a magnesium salt of gamma-hydroxybutyrate (Mg.(GHB)₂), and a calcium salt of gamma-hydroxybutyrate (Ca.(GHB)₂). In some embodiments, the mixed salt oxybate comprises varying weight/weight percentages (wt/wt %) of Na.GHB, K.GHB, Mg.(GHB)₂, and Ca.(GHB)₂.

In some embodiments, any of the salts, such as the Na.GHB salt, the K.GHB salt, the Mg.(GHB)₂ salt or the Ca.(GHB)₂, is present in about 1%-5%, about 5%-10%, about 10% -15%, about 15%-20%, about 20%-25%, about 25%-30%, about 30%-35%, about 35%-40%, about 40%-45%, about 45%-50%, about 50%-55%, about 55%-60%, about 60%-65%, about 65%-70%, about 70%-75%, about 75%-80%, about 80%-85%, about 85%-90%, about 90%-95%, or about 95%-100% (wt/wt %). In some embodiments, the Na.GHB salt is present in a wt/wt % of about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% (wt/wt %). In some embodiments, the Na.GHB salt is absent.

In some embodiments, where the mixed salt oxybate comprises a mixture of Na.GHB, K.GHB, Mg.(GHB)₂, and Ca.(GHB)₂, the Na.GHB salt is present in a wt/wt % of about 1%-15%, 5%-10%, or about 8%; the K.GHB salt is present in a wt/wt % of about 10%-30%, 15%-25%, or about 25.5%; the Mg.(GHB)₂ salt is present in a wt/wt % of about 10%-30%, 15%-25%, or about 19.5%; and the Ca.(GHB)₂ salt is present in a wt/wt % of about 30%-60%, 40%-50, or about 47% (wt/wt %).

In some embodiments, the mixed salt oxybate comprises about 8% of sodium oxybate (wt/wt %), about 25.5% of potassium oxybate (wt/wt %), about 19.5% of magnesium oxybate (wt/wt %) and about 47% of calcium oxybate (wt/wt %). In some embodiments, where the mixed salt oxybate comprises a mixture of Na.GHB, K.GHB, Mg.(GHB)₂, and Ca.(GHB)₂, the Na.GHB, K.GHB, Mg.(GHB)₂, and Ca.(GHB)₂ salts are present in a wt/wt % ratio of about 8:25.5:19.5:47, respectively.

In some embodiments, a mixed salt oxybate of the present disclosure is dissolved in a liquid (such as water) to provide a pharmaceutical composition and the concentration of the mixed salt oxybate is expressed in terms of the wt/vol %. In some embodiments, where the mixed salt oxybate comprises a mixture of Na.GHB, K.GHB, Mg.(GHB)₂, and Ca.(GHB)₂, the Na.GHB salt is present in a wt/vol % of about 1%-15%, 5%-10%, or about 8%; the K.GHB salt is present in a wt/vol % of about 10%-30%, 15%-25%, or about 26%; the Mg.(GHB)₂ salt is present in a wt/vol % of about 10%-30%, 15%-25%, or about 19.2%; and the Ca.(GHB)₂ salt is present in a wt/vol % of about 30%-60%, 40%-50, or about 46.8% (wt/vol %).

In some embodiments, the liquid pharmaceutical composition containing the mixed salt oxybate comprises about 8% of sodium oxybate (wt/vol %), about 26.0% of potassium oxybate (wt/vol %), about 19.2% of magnesium oxybate (wt/vol %) and about 46.8% of calcium oxybate (wt/vol %).

In some embodiments, the mixed salt oxybate comprises varying percentages of oxybate, expressed as % molar equivalents (% mol. equiv.) of Na.GHB, K.GHB, Mg.(GHB)₂, and Ca.(GHB)₂. The terms “% molar equivalents” and “% mol. equiv.,” as used herein, refer to molar composition of salts expressed as a percent of GHB equivalents. Those skilled in the art will understand that as each GHB unit is considered to be one molar equivalent, the monovalent cations, Na⁺ and K⁺, have one molar equivalent per salt, and the divalent cations, Mg⁺² and Ca⁺², have two molar equivalents per salt. See U.S. Pat. Nos. 8,591,922; 8,901,173; 9,132,107; 9,555,017; 10,195,168 for amounts of % mol. equiv. useful in the present disclosure.

In some embodiments, any of the salts, such as the Na.GHB salt, the K.GHB salt, the Mg.(GHB)₂ salt or the Ca.(GHB)₂, is present in about 1%-5%, about 5%-10%, about 10%-15%, about 15%-20%, about 20%-25%, about 25%-30%, about 30%-35%, about 35%-40%, about 40%-45%, about 45%-50%, about 50%-55%, about 55%-60%, about 60%-65%, about 65%-70%, about 70%-75%, about 75%-80%, about 80%-85%, about 85%-90%, about 90%-95%, or about 95%-100% (% mol. equiv.). In some embodiments, the Na.GHB salt is present in a % mol. equiv. of about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% (% mol. equiv.). In some embodiments, the Na.GHB salt is absent.

In some embodiments, where the mixed salt oxybate comprises a mixture of Na.GHB, K.GHB, Mg.(GHB)₂, and Ca.(GHB)₂, the Na.GHB salt is present in a % mol. equiv. of about 1%-15%, 5%-10%, or about 8%; the K.GHB salt is present in a % mol. equiv. of about 10%-30%, 15%-25%, or about 23%; the Mg.(GHB)₂ salt is present in a % mol. equiv. of about 10%-30%, 15%-25%, or about 21%; and the Ca.(GHB)₂ salt is present in a % mol. equiv. of about 30%-60%, 40%-50, or about 48% (% mol. equiv.).

In some embodiments, the mixed salt oxybate comprises about 8% mol. equiv. of sodium oxybate, about 23% mol. equiv. of potassium oxybate, about 21% mol. equiv. of magnesium oxybate and about 48% mol. equiv. of calcium oxybate. In some embodiments, where the mixed salt oxybate comprises a mixture of Na.GHB, K.GHB, Mg.(GHB)₂, and Ca.(GHB)₂, wherein the mixture comprises Na.GHB, K.GHB, Mg.(GHB)₂, and Ca.(GHB)₂ salts are present in a % mol. equiv. ratio of about 8:23:21:48, respectively.

In some embodiments, where the pharmaceutical composition comprises a mixture of Na.GHB, K.GHB, and Ca.(GHB)₂, the Na.GHB salt is present in a % mol. equiv. of about 5%-40%, the K.GHB salt is present in a % mol. equiv. of about 10%-40%, and the Ca.(GHB)₂ salt is present in a % mol. equiv. of about 20%-80%.

### Pharmaceutical Compositions:

In some embodiments, the mixed salt oxybate is in the form of a pharmaceutical composition that is suitable for administration in the methods of the present disclosure.

In some embodiments, the pharmaceutical composition comprises an aqueous solution.

In some embodiments, the concentration of the mixture of salts of GHB in the solution is about 50 mg/mL-950 mg/mL, about 250 mg/mL-750 mg/mL, about 350 mg/mL-650 mg/mL, or about 450 mg/mL-550 mg/mL. In some embodiments, the concentration of the mixture of salts of GHB in the solution is about 500 mg/mL.

In some embodiments, the pH of the pharmaceutical composition is about 7.0-9.0, about 7.0-8.5, or about 7.3-8.5.

In some embodiments, the pharmaceutical composition is chemically stable and resistant to microbial growth. In some embodiments, the pharmaceutical composition is free of preservatives. See U.S. Pat. Nos. 6,472,431; 6,780,889; 7,262,219; 8,263,650; 8,461,203 and others for a relationship between pH and GHB concentration and their effect on microbial growth.

In some embodiments, a pH adjusting or buffering agent may be added to the pharmaceutical composition. The choice of a pH adjusting or buffering agent may affect the resistance to microbial challenge and/or the stability of GHB, as measured by the reduction in assayable GHB. Pharmaceutical compositions of GHB, pH adjusted or buffered with malic acid are resistant to both microbial growth and chemical degradation of GHB, and are preferred. Other pH adjusting or buffering agents may be selected. Agents that adjust pH that are selected on this basis will undergo a taste testing study. However, any pH adjusting or buffering agent disclosed herein or as would be known to those skilled in the art is contemplated as being useful from the compositions or formulations disclosed herein. Of course, any salt, flavoring agent, excipient, or other pharmaceutically acceptable addition described herein or as would be known to those skilled in the art is contemplated as being useful for the compositions or formulations disclosed herein.

In some embodiments, the pH adjusting or buffering agent is an acid. In some embodiments, the pH adjusting or buffering agent is an inorganic acid or an organic acid. In some embodiments, the pH adjusting or buffering agent is selected from the group consisting of malic acid, citric acid, acetic acid, boric acid, lactic acid, hydrochloric acid, phosphoric acid, sulfuric acid, sulfonic acid, and nitric acid. In some embodiments, the pH adjusting or buffering agent is malic acid.

The aqueous solutions disclosed herein typically comprise an effective amount of GHB, which may be dissolved or dispersed in a pharmaceutically acceptable carrier and/or an aqueous medium.

### Formulations

In some embodiments, the pharmaceutical compositions disclosed herein are provided in a formulation that is suitable for administration in the methods of the present disclosure.

In some embodiments, the formulation is a liquid formulation. In some embodiments, the formulation is a solid formulation. See incorporated by reference U.S. Pat. Nos. 6,472,431; 6,780,889; 7,262,219; 8,263,650; 8,461,203, 8,591,922, 8,901,173, 9,132,107, 9,555,017, 9,795,567, 10,195,168, U.S. Ser. Nos. 62/769,380 and 62/769,382 and U.S. Patent Publication No. 2018/0263936 for example.

In some embodiments, the formulation is chemically stable and resistant to microbial growth. In some embodiments, the formulation is free of preservatives. In some embodiments, the level of gamma-butyrolactone (GBL) is 0.1% or less of the formulation. In some embodiments, the level of gamma-butyrolactone (GBL) is 0.5% or less of the formulation.

In some embodiments, the formulation is suitable for oral administration. See incorporated by reference U.S. Pat. Nos. 6,472,431; 6,780,889; 7,262,219; 8,263,650; 8,461,203, 8,591,922, 8,901,173, 9,132,107, 9,555,017, and 10,195,168 and U.S. Ser. Nos. 62/769,380 and 62/769,382 for examples of flavoring agents, sweeteners, coloring agents, surfactants, carriers, excipients, binders, buffering compounds or agents and other formulation ingredients.

In preferred embodiments, the formulation is a liquid formulation, wherein the formulation comprises 0.234 g/mL of calcium oxybate, 0.130 g/mL of potassium oxybate, 0.096 g/mL of magnesium oxybate, and 0.040 g/mL of sodium oxybate (which contains 0.409 g/mL of GHB or Equivalent Concentration of GBA of 0.413 g/mL).

In some embodiments, the formulation is suitable for administration in a single or multiple dosage regimen. See U.S. Ser. Nos. 62/769,380 and 62/769,382.

Any of the above formulations may be prepared and/or packaged as a powdered or dry form for mixing with an aqueous medium before oral administration, or they may be prepared in an aqueous medium and packaged. After mixing with an aqueous medium, preferably to prepare a solution, these formulations are resistant to both microbial growth and chemical conversion of GHB to GBL, thereby increasing the shelf-life of therapeutic formulations of GHB in an aqueous medium. These formulations then provide an easily titratable liquid medium for measuring the dosage of GHB to be administered to a patient.

The GHB may be lyophilized for more ready formulation into a desired vehicle or medium where appropriate. The active compounds may be formulated for parenteral administration, e.g., formulated for injection via intravenous, intraarterial, intramuscular, sub-cutaneous, intralesional, intraperitoneal or other parenteral routes. The preparation of a composition that comprises an aqueous solution that contains a GHB agent as an active component or ingredient will be known to those of skill in the art in light of the present disclosure. Typically, such compositions can be prepared as injectables, either as liquid solutions or suspensions. Solid forms suitable for using to prepare solutions or suspensions upon the addition of a liquid prior to injection can also be prepared; and the preparations can also be emulsified. See U.S. Pat. Nos. 6,472,431; 6,780,889; 7,262,219; 8,263,650; 8,461,203, 8,591,922, 8,901,173, 9,132,107, 9,555,017, 9,795,567, 10,195,168, U.S. Ser. Nos. 62/769,380 and 62/769,382, and U.S. Patent Publication No. 2018/0263936 for example for more information about parenteral administration.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For oral therapeutic administration, the active compounds may be incorporated with excipients and used in the form of tablets, buccal tablets or tabs, troches, capsules, elixirs, suspensions, syrups, wafers, and the like, to be admixed with an aqueous medium. Such compositions and preparations should contain at least 0.1% of active compound. The percentage of the compositions and preparations may, of course, be varied and may conveniently be between about 2-75% of the weight of the unit, or preferably between 25-60%. The amount of active compounds in such therapeutically useful compositions is such that a suitable dosage will be obtained. See U.S. Pat. Nos. 6,472,431; 6,780,889; 7,262,219; 8,263,650; 8,461,203, 8,591,922, 8,901,173, 9,132,107, 9,555,017, 9,795,567, 10,195,168, U.S. Ser. Nos. 62/769,380 and 62/769,382, and U.S. Patent Publication No. 2018/0263936 for example.

### Methods of the Present Disclosure

In one aspect, the present disclosure provides methods of substituting, exchanging, changing or switching a mixed salt oxybate composition for a sodium oxybate composition in a patient treated with sodium oxybate (such as a patient treated for cataplexy or excessive daytime sleepiness in patients with narcolepsy), wherein the amount of sodium oxybate and mixed salt oxybate are the same on an oxybate dosing strength basis.

In one aspect, the present disclosure provides methods for changing or switching a patient who is administered sodium oxybate to a mixed salt oxybate composition, the method comprising: administering a therapeutically effective amount a mixed salt oxybate to the patient, wherein the amount of sodium oxybate and the mixed salt oxybate are the same on an oxybate dosing strength basis.

In one aspect, the present disclosure provides methods for treating a patient for a condition that is treated by sodium oxybate, the method comprising:
Switching or changing the dose of a patient who is administered sodium oxybate to a mixed salt oxybate,
wherein the switching comprises administering a therapeutically effective amount of the mixed salt oxybate to the patient and wherein the amount of sodium oxybate and mixed salt oxybate are the same on an oxybate dosing strength basis.

In some embodiments, the substitution, exchange, change or switch from sodium oxybate to the mixed salt oxybate occurs in successive doses (i.e., sodium oxybate is administered in a first dose and a mixed salt oxybate is administered in same amount on an oxybate dosing strength basis in the next consecutive dose). In some embodiments, the patient is administered sodium oxybate on one day and the mixed salt oxybate is administered in same amount on an oxybate dosing strength basis on the next day. In some embodiments, the methods of the present disclosure comprise administering two oxybate doses per day, wherein the first dose consists of sodium oxybate (e.g., Xyrem®) and the second dose consists of a mixed salt oxybate.

In some embodiments, the present disclosure provides methods of substituting, exchanging, changing or switching a mixed salt oxybate composition for a sodium oxybate composition in a patient treated for cataplexy or excessive daytime sleepiness in patients with narcolepsy, the method comprising:
a. Determining whether a patient treated with a therapeutically effective amount of sodium oxybate is sensitive to high sodium intake; and
b. If the patient is sensitive to high sodium intake, then administering a therapeutically effective amount of a mixed salt oxybate to the patient,
wherein the amount of the sodium oxybate and mixed salt oxybate are the same on a oxybate dosing strength basis.

In some embodiments, present disclosure provides methods for a 1-to-1 dose switch from sodium oxybate (such as Xyrem®) to a mixed salt oxybate.

In some embodiments, the substitution, exchange, change or switch from sodium oxybate to a mixed salt oxybate comprises administering a mixture of the two oxybate formulations (e.g. Xyrem® and a mixed salt oxybate of the present disclosure) during the transition period. In some embodiments, the transition period is less than about one week, about two weeks, about three weeks, about four weeks or about five weeks. In some embodiments, the transition period is about one week, about two weeks, about three weeks, about four weeks or about five weeks.

According to the methods of the present disclosure, the mixed salt oxybate that is administered may be any of the mixed salt oxybate compositions described herein. In some embodiments, the relative amount of each salt in the mixed salt oxybate that is administered is expressed in terms of wt/wt %. In some embodiments, the mixed salt oxybate comprises sodium oxybate, potassium oxybate, magnesium oxybate and calcium oxybate, and wherein the mixed salt oxybate comprises about 5%-40% of sodium oxybate (wt/wt %). In some embodiments, the mixed salt oxybate comprises about 5%-40% of sodium oxybate (wt/wt %), about 10%-40% of potassium oxybate (wt/wt %), about 5%-30% of magnesium oxybate (wt/wt %), and about 20%-80% of calcium oxybate (wt/wt %). In some embodiments, the mixed salt oxybate comprises about 8% of sodium oxybate (wt/wt %), about 25.5% of potassium oxybate (wt/wt %), about 19.5% of magnesium oxybate (wt/wt %) and about 47% of calcium oxybate (wt/wt %).

In some embodiments, the relative amount of each salt in the mixed salt oxybate that is administered in a liquid pharmaceutical composition is expressed in terms of wt/vol %. In some embodiments, the liquid pharmaceutical composition comprises a mixed salt oxybate comprising sodium oxybate, potassium oxybate, magnesium oxybate and calcium oxybate, and wherein the mixed salt oxybate comprises about 5%-40% of sodium oxybate (wt/vol %). In some embodiments, the liquid pharmaceutical composition comprises a mixed salt oxybate comprising about 5%-40% of sodium oxybate (wt/vol %), about 10%-40% of potassium oxybate (wt/vol %), about 5%-30% of magnesium oxybate (wt/vol %), and about 20%-80% of calcium oxybate (wt/vol %). In some embodiments, the liquid pharmaceutical composition comprises the mixed salt oxybate comprising about 8% of sodium oxybate (wt/vol %), about 26% of potassium oxybate (wt/vol %), about 19.2% of magnesium oxybate (wt/vol %) and about 46.8% of calcium oxybate (wt/vol %).

In some embodiments, the relative amount of each salt in the mixed salt oxybate that is administered is expressed in terms of % mol. equiv. In some embodiments, the mixed salt oxybate comprises sodium oxybate, potassium oxybate, magnesium oxybate and calcium oxybate, and wherein the mixed salt oxybate comprises about 5%-40% mol. equiv. of sodium oxybate. In some embodiments, the mixed salt oxybate comprises about 5%-40% mol. equiv. of sodium oxybate, about 10%-40% mol. equiv. of potassium oxybate, about 5%-30% mol. equiv. of magnesium oxybate, and about 20%-80% mol. equiv. of calcium oxybate. In some embodiments, the mixed salt oxybate comprises about 8% mol. equiv. of sodium oxybate, about 23% mol. equiv. of potassium oxybate, about 21% mol. equiv. of magnesium oxybate and about 48% mol. equiv. of calcium oxybate.

In some embodiments, the mixed salt oxybate is administered twice per day. In some embodiments, the mixed salt oxybate is administered once per day, See U.S. Ser. Nos. 62/769,380 and 62/769,382. In some embodiments, the mixed salt oxybate is administered at bedtime. In some embodiments, the mixed salt oxybate is administered at bedtime and about 2.5 h-4 h after the bedtime administration.

In some embodiments, the dose of the mixed salt oxybate is described in terms of the amount of the mixed salt oxybate that is administered to the patient. In some embodiments, about 0.25 g-10.0 g, about 1.0 g-9.0 g, about 2.0 g-10.0 g; about 3.0 g-9.5 g; or about 4.5 g-9.0 g of the mixed salt oxybate is administered per day.

In some embodiments, about 1.0 g of the mixed salt oxybate (such as JZP-258) is administered per day. In some embodiments, about 0.5 g of the mixed salt oxybate (such as JZP-258) is administered twice per day. In some embodiments, about 3.0 g of the mixed salt oxybate (such as JZP-258) is administered per day. In some embodiments, about 1.5 g of the mixed salt oxybate (such as JZP-258) is administered twice per day. In some embodiments, about 4.5 g of the mixed salt oxybate (such as JZP-258) is administered per day. In some embodiments, about 2.25 g of the mixed salt oxybate (such as JZP-258) is administered twice per day. In some embodiments, wherein about 6.0 g of the mixed salt oxybate (such as JZP-258) is administered per day. In some embodiments, about 3.0 g of the mixed salt oxybate (such as JZP-258) is administered twice per day. In some embodiments, about 7.5 g of the mixed salt oxybate (such as JZP-258) is administered per day. In some embodiments, wherein about 3.75 g of the mixed salt oxybate (such as JZP-258) is administered twice per day. In some embodiments, about 9.0 g of the mixed salt oxybate (such as JZP-258) is administered per day. In some embodiments, about 4.5 g of the mixed salt oxybate (such as JZP-258) is administered twice per day.

In some embodiments, the dose of the mixed salt oxybate is described in terms of the amount of GHB that is administered to the patient. In some embodiments, a mixed salt oxybate (such as JZP-258) containing about 0.818 g-7.362 g, about 1.636 g-8.18 g; about 2.454 g-7.771 g; or about 3.681 g-7.362 g of GHB is administered per day.

In some embodiments, a mixed salt oxybate (such as JZP-258) containing about 0.818 g of GHB is administered per day. In some embodiments, a mixed salt oxybate (such as JZP-258) containing about 0.409 g of GHB is administered twice per day. In some embodiments, a mixed salt oxybate (such as JZP-258) containing about 2.454 g of GHB is administered per day. In some embodiments, a mixed salt oxybate (such as JZP-258) containing about 1.227 g of GHB is administered twice per day. In some embodiments, a mixed salt oxybate (such as JZP-258) containing about 3.681 g of GHB is administered per day. In some embodiments, a mixed salt oxybate (such as JZP-258) containing about 1.841 g of GHB is administered twice per day. In some embodiments, a mixed salt oxybate (such as JZP-258) containing about 4.908 g of GHB is administered per day. In some embodiments, a mixed salt oxybate (such as JZP-258) containing about 2.454 g of GHB is administered twice per day. In some embodiments, a mixed salt oxybate (such as JZP-258) containing about 6.135 g of GHB is administered per day. In some embodiments, a mixed salt oxybate (such as JZP-258) containing about 3.068 g of GHB is administered twice per day. In some embodiments, a mixed salt oxybate (such as JZP-258) containing about 7.362 g of GHB is administered per day. In some embodiments, a mixed salt oxybate (such as JZP-258) containing about 3.681 g of GHB is administered twice per day.

Although throughout the present disclosure, the amount of oxybate administered in a composition is generally expressed in terms of the amount of GHB administered (see above), the present disclosure contemplates embodiments where the oxybate dosing is expressed in the Equivalent Amount of GBA that is administered.

The Equivalent Amount of GBA in a compositions may be calculated by the following formula:

$$Equivalent Amount of GBA = \\ \frac{Amount of GHB in ( g ) \times 104.1 ( Formula Weight of GBA , \frac{g}{mol} )}{103.1 ( Formula Weight of GHB ( \frac{g}{mol} )}$$

In some embodiments, the dose of the mixed salt oxybate is described in terms of the amount of Equivalent Amount of GBA that is administered to the patient. In some embodiments, a mixed salt oxybate (such as JZP-258) containing about 0.826 g-7.434 g, about 1.652 g-8.26 g; about 2.478 g-7.847 g; or about 3.717 g-7.434 g of an Equivalent Amount of GBA is administered per day.

In some embodiments, a mixed salt oxybate (such as JZP-258) containing about 0.826 g of an Equivalent Amount of GBA is administered per day. In some embodiments, a mixed salt oxybate (such as JZP-258) containing about 0.413 g of an Equivalent Amount of GBA is administered twice per day. In some embodiments, a mixed salt oxybate (such as JZP-258) containing about 2.478 g of an Equivalent Amount of GBA is administered per day. In some embodiments, a mixed salt oxybate (such as JZP-258) containing about 1.239 g of an Equivalent Amount of GBA is administered twice per day. In some embodiments, a mixed salt oxybate (such as JZP-258) containing about 3.717 g of an Equivalent Amount of GBA is administered per day. In some embodiments, a mixed salt oxybate (such as JZP-258) containing about 1.859 g of an Equivalent Amount of GBA is administered twice per day. In some embodiments, a mixed salt oxybate (such as JZP-258) containing about 4.956 g of an Equivalent Amount of GBA is administered per day. In some embodiments, a mixed salt oxybate (such as JZP-258) containing about 2.478 g of an Equivalent Amount of GBA is administered twice per day. In some embodiments, a mixed salt oxybate (such as JZP-258) containing about 6.195 g of an Equivalent Amount of GBA is administered per day. In some embodiments, a mixed salt oxybate (such as JZP-258) containing about 3.098 g of an Equivalent Amount of GBA is administered twice per day. In some embodiments, a mixed salt oxybate (such as JZP-258) containing about 7.434 g of an Equivalent Amount of GBA is administered per day. In some embodiments, a mixed salt oxybate (such as JZP-258) containing about 3.717 g of an Equivalent Amount of GBA is administered twice per day.

In some embodiments, the methods provided herein for substitution, exchange, change or switch from sodium oxybate to a mixed salt oxybate further comprise reducing the dose of the mixed salt oxybate by at least about 20% when the patient is co-administered divalproex sodium.

In some embodiments, the methods of the present disclosure comprise oral administration of the compositions or formulations comprising a mixed salt oxybate (disclosed herein) in a multiple dosage regimen. See U.S. Pat. No. 8,591,922, which is hereby incorporated by reference in its entirety for all purposes. In some embodiments, the multiple dosage regimen comprises one or more steps, as follows: (i) diluting an aqueous solution comprising about 500 mg/mL of the mixed salt oxybate with an aqueous medium to provide a first dose of about 1-10 grams of the mixture of salts; (ii) orally administering the dose to a patient; (iii) diluting an aqueous solution comprising about 500 mg/mL of the mixed salt oxybate to provide a second dose of about 1-10 grams of the mixed salt oxybate; and (iv) orally administering to the patient the second dose. The dose administered to the patient can be between about 2.25-4.5 grams. (All volumes and numbers are presented as Na GHB equivalents).

In the majority of patients, the substitution, exchange, change or switch from sodium oxybate to a mixed salt oxybate is a gram for gram substitution wherein the amount of GHB administered in the sodium oxybate and mixed salt oxybate doses is the same. However, in some instances, a small dose adjustment (or titration) is need after switching the dose. In some embodiments, on the first night of dosing with the mixed salt oxybate (e.g., JZP-258), treatment is initiated at the same dose (gram for gram) and regimen as sodium oxybate, and titrated as needed based on efficacy and tolerability. In some embodiments, the method of the present disclosure further comprises titrating the dose of the mixed salt oxybate after the substituting, exchanging, changing or switching. In some embodiments, the titration period is from 1 day to 8 weeks, 1 week to 6 weeks, or 2 weeks to 4 weeks. The titration period can be about 1 day, 2 days, 3 days, 4 days, 5 days, 6 days, 1 week, 2 weeks, 3 weeks, 4 weeks, 5 weeks, 6 weeks, 7 weeks or 8 weeks.

In some embodiments, the titration comprises increasing the daily dose of the mixed salt oxybate compared to the daily dose of sodium oxybate. In some embodiments, the titration comprises increasing the daily dose by less than about 1.5 g of the mixed salt oxybate compared to the daily dose of sodium oxybate. In some embodiments, the titration comprises increasing the daily dose by about 0.25 g, about 0.5 g, about 1.0 g, about 1.5 g, or about 2.0 g of the mixed salt oxybate compared to the daily dose of sodium oxybate. In some embodiments, the titration comprises increasing the daily dose by about 1.0 g-1.5 g of the mixed salt oxybate compared to the daily dose of sodium oxybate.

In some embodiments, the titration comprises decreasing the daily dose of the mixed salt oxybate compared to the daily dose of sodium oxybate. In some embodiments, the titration comprises decreasing the daily dose by less than about 1.5 g of the mixed salt oxybate compared to the daily dose of sodium oxybate. In some embodiments, the titration comprises decreasing the daily dose by about 0.25 g, about 0.5 g, about 0.75 g, about 1.0 g, about 1.25 g, about 1.5 g, about 1.75 g, or about 2.0 g of the mixed salt oxybate compared to the daily dose of sodium oxybate. In some embodiments, the titration comprises decreasing the daily dose by about 1.0 g-1.5 g of the mixed salt oxybate compared to the daily dose of sodium oxybate.

In some embodiments, the patient switched from sodium oxybate to a mixed salt oxybate composition (gram for gram) is an adult patient. In some embodiments, the patient switched from oxybate to a mixed salt oxybate composition (gram for gram) is a pediatric patient.

In some embodiments, the present disclosure provides methods of transitioning from sodium oxybate to a mixed salt oxybate composition, wherein the mixed salt oxybate is administered with food. In some embodiments, the mixed salt oxybate composition is administered without food. In some embodiments, the mixed salt oxybate composition is administered with or without regard to food. In some embodiments, the patient is administered the mixed salt oxybate composition at least 2 h after the patient's last meal. In some embodiments, the patient is administered their first dose of the mixed salt oxybate composition (i.e., the dose where the patient transitions from sodium oxybate to the mixed salt oxybate composition) at least 2 h after the patient's last meal. In some embodiments, the patient is administered their first dose of the mixed salt oxybate composition at least 2 h, at least 1.5 h, about 1.0 h, about 0.5 h or about 15 min after the patient's last meal. In some embodiments, the mixed salt oxybate is administered with or without regard to food after the titration period as described herein (i.e., when a stable dose of the mixed salt oxybate composition is achieved).

The embodiments are described in terms of administering a mixed salt oxybate composition; however, the present disclosure also contemplates the administration of the mixed salt oxybate in the compositions and formulations described herein. In some embodiments, the mixed salt oxybate composition is a liquid. In some embodiments, the concentration of the mixed salt in the liquid is from 50 mg/mL-950 mg/mL, about 250 mg/mL-750 mg/mL, about 350 mg/mL-650 mg/mL, or about 450 mg/mL-550 mg/mL. In some embodiments, the concentration of the mixed salt in the liquid is about 0.5 g/mL.

In some embodiments, the patient administered the mixed salt oxybate is a patient at risk for the undesirable side effects related to high sodium intake. In some embodiments, the patient is in heart failure. In some embodiments, the patient is hypertensive. In some embodiments, the patient has renal impairment. In some embodiments, the patient is at risk for stroke.

In some embodiments, the patient administered the mixed salt oxybate is a patient with hepatic impairment. In some embodiments, the hepatic impairment of the patient administered the mixed salt oxybate is determine by the Child Pugh Classification for Severity of Liver Disease. The Child Pugh Classification for Severity of Liver Disease is a 15 point scale that assesses the severity of hepatic impairment. The presence of encephalopathy, ascites, concentration of bilirubin and albumin, and prothrombin time prolongation are assessed in the Child Pugh Classification for Severity of Liver Disease. A patient with hepatic impairment that is assigned a score of 5 to 6 points on the Child Pugh Classification for Severity of Liver Disease is assigned to Child Class A. A patient with hepatic impairment that is assigned a score of 7 to 9 points on the Child Pugh Classification for Severity of Liver Disease is assigned to Child Class B. A patient with hepatic impairment that is assigned a score of 10 to 15 points on the Child Pugh Classification for Severity of Liver Disease is assigned to Child Class C.

In some embodiments, the patient administered the mixed salt oxybate is a patient in Child Class A, Child Class B, or Child Class C. In some embodiments, the patient administered the mixed salt oxybate is a patient in Child Class A. In some embodiments, the patient administered the mixed salt oxybate is a patient in Child Class B. In some embodiments, the patient administered the mixed salt oxybate is a patient in Child Class C.

In some embodiments, patients with hepatic impairment treated according to the methods of the present disclosure are administered one-half of the initial dose of mixed salt oxybate that is recommended for a patient without hepatic impairment. In some embodiments, patients with hepatic impairment treated according to the methods of the present disclosure are administered between 40% to 60% of the initial dose of mixed salt oxybate that is recommended for a patient without hepatic impairment. In some embodiments, patients with hepatic impairment treated according to the methods of the present disclosure are administered an initial dose of mixed salt oxybate that is less than the dose recommended for a patient without hepatic impairment.

In some embodiments, the patient with hepatic impairment that would receive an initial dose of about 1.0 g of the mixed salt oxybate (such as JZP-258) in the absence of hepatic impairment is administered about 0.5 g per day of the mixed salt oxybate (such as JZP-258). In some embodiments, the patient with hepatic impairment that would receive an initial dose of about 1.0 g of the mixed salt oxybate (such as JZP-258) in the absence of hepatic impairment is administered about 0.25 g of the mixed salt oxybate (such as JZP-258) twice per day.

In some embodiments, the patient with hepatic impairment that would receive an initial dose of about 1.5 g of the mixed salt oxybate (such as JZP-258) in the absence of hepatic impairment is administered about 0.75 g per day of the mixed salt oxybate (such as JZP-258). In some embodiments, the patient with hepatic impairment that would receive an initial dose of about 1.5 g of the mixed salt oxybate (such as JZP-258) in the absence of hepatic impairment is administered about 0.38 g of the mixed salt oxybate (such as JZP-258) twice per day.

In some embodiments, the patient with hepatic impairment that would receive an initial dose of about 2.25 g of the mixed salt oxybate (such as JZP-258) in the absence of hepatic impairment is administered about 1.13 g per day of the mixed salt oxybate (such as JZP-258). In some embodiments, the patient with hepatic impairment that would receive an initial dose of about 2.25 g of the mixed salt oxybate (such as JZP-258) in the absence of hepatic impairment is administered about 0.56 g of the mixed salt oxybate (such as JZP-258) twice per day.

In some embodiments, the patient is treated for a sleep disorder such as apnea, sleep time disturbances, narcolepsy, cataplexy, sleep paralysis, hypnagogic hallucination, sleep arousal, insomnia, and nocturnal myoclonus. In some embodiments, the patient is treated for cataplexy. In some embodiments, the patient is treated is treated for excessive daytime sleepiness in patients with narcolepsy. In some embodiments, the patient is treated is treated for excessive daytime sleepiness in patients with idiopathic hypersomnia. See U.S. Pat. Nos. 6,472,431; 6,780,889; 7,262,219; 8,263,650; 8,461,203, 8,591,922, 8,901,173, 9,132,107, 9,555,017, 9,795,567, 10,195,168, U.S. Ser. Nos. 62/769,380 and 62/769,382, and U.S. Patent Publication No. 2018/0263936 for example.

### Methods of Making

The mixed salt oxybate, compositions and formulations may be prepared using methods that are known to those skilled in the art, including the methods described U.S. Pat. Nos. 8,591,922; 8,901,173; 9,132,107; 9,555,017; 10,195,168 and U.S. Publication No. 2018/0263936, which are hereby incorporated by reference).

### EXAMPLES

### Example 1

Two Phase 1 bioequivalence/bioavailability (BE/BA) studies were performed in healthy volunteers to characterize the pharmacokinetics (PK) of JZP-258.

Study 1: An Open-Label, Randomized Crossover Study to Evaluate the Pharmacokinetics, Bioavailability, Bioequivalence, and Food Effect Following Administration of Oxybate Formulations.

Primary Objectives: (1) To assess the relative bioavailability and bioequivalence of JZP-258 compared with Xyrem oral solution under fasting and fed conditions (2) To evaluate the PK of JZP-258 under fasting and fed conditions (food effect) (3) To evaluate the relative bioavailability and bioequivalence of two admixtures of JZP-258 and Xyrem at different ratios compared with Xyrem oral solution under fasting conditions (4) To evaluate the PK of JZP-258 2.25 g under fasting conditions.

Part 1: Subjects were randomized into four groups and treated either 4.5 g of Xyrem or 4.5 g of JZP-258 under fasting or fed conditions.

Part 2: Admixtures of JZP-258 and Xyrem in different ratios were compared to Xyrem under fasting conditions.

Evaluation: Blood samples to determine oxybate PK profiles were to be collected predose; at 10, 20, 30, 45, 60, and 75 minutes postdose; and at 1.5, 2, 2.5, 3, 3.5, 4, 4.5, 5, 6, 7, and 8 hours postdose following each treatment on Days 1, 3, 5, and 7. Blood samples for PK analysis were then obtained within ±2 minutes of the specified time points for the first hour after each dose and within ±5 minutes of the specified time points after one hour. The actual time of blood sample collection was recorded. A minimum 1-day washout period was to separate the four treatments.

Data Collection: The PK parameters calculated for plasma oxybate included Cₘₐₓ, Tₘₐₓ, t_{1/2}, λ_{z}, AUC₀₋ₜ, and AUC_{0-inf}.

TABLE 1
Study Design
Treatment Periods^{a}
Study Days
Dosing Sschedule

| | Screening | Baseline | | | | | |
|---|---|---|---|---|---|---|---|
| | Period | Period | Period 1 | Period 2 | Period3 | Period 4 | Final Day |
| | Days −21 to −2 | Day −1 | Days 1 to 2 | Days 3 to 4 | Days 5 to 6 | Days 7 to 8 | Day 8 |
| Part 1^{b} | | | Treatments | Treatments | Treatments | Treatments | |
| | | | A, B, C, or D | A, B, C, or D | A, B, C, or D | A, B, C, or D | |
| Part 2^{b} | | | Treatments | Treatments | Treatments | Treatments | |
| | | | E, F, G, or H | E, F, G, or H | E, F, G, or H | E, F, G, or H | |

^{a}A 1-day washout seperated each of the four treatments.
^{b}Subjects were randomized to one of four sequences to receive Treatments A, B, C, and D in Part 1, and Treatments E, F, G, and H in Part 2.
Note:
In parts 1: Treatment A = 4.5 g JZP-258 under fasting conditions; Treatment B = 4.5 g Xyrem under fed conditions. In Part 2: Treatment E = Admixture of JZP-258 2.2 g, and 2 g Xyrem, under fasting conditions (total of 4.5 g oxybate); Treatment F = Admixture of JZP-258 3.75 g, and 0.75 g Xyrem, under fasting conditions (total of 4.5 g oxybate); Treatment G = 4.5 g Xyrem, under fasting conditions; and Treatment H = 2.25 g JZP-258, under fasting conditions.

Study 2 (JZP258-101): An Open-Label, Randomized Crossover, Phase 1 Study to Evaluate the Pharmacokinetics, Bioavailability, and Bioequivalence Following Administration of Oxybate Formulations in Healthy Subjects.

Primary Objective: To assess the relative bioavailability and bioequivalence of JZP-258 oral solution versus Xyrem taken with 60 mL water under fasting conditions.

Subjects were randomized into six groups and treated either 4.5 g of Xyrem or 4.5 g of JZP-258 under fasting of fed conditions taken with 60 mL of water 240 mL.

Blood samples to determine oxybate PK profiles were to be collected predose; at 10, 20, 30, 45, 60, and 75 minutes postdose; and at 1.5, 2, 2.5, 3, 3.5, 4, 4.5, 5, 6, 7, and 8 hours postdose following each dose on Days 1, 3, 5, 7, 9, and 11. Blood samples for PK analysis were to be taken within ±2 minutes of the specified time points for the first hour after each dose and within ±5 minutes of the specified time points after one hour. The actual time of blood sample collection was recorded. A minimum 1-day washout period separated the six treatments.

TABLE 2
Study Design
Treatment Periods^{a}
Study Days
Dosing Schedule

| Screening | Baseline | | | | | | | Final |
|---|---|---|---|---|---|---|---|---|
| Period | Period | Period 1 | Period 2 | Period 3 | Period 4 | Period 5 | Period 6 | Day |
| Days −21 to −2 | Day −1 | Days 1 to 2 | Days 3 to 4 | Days 5 to 6 | Days 7 to 8 | Days 9 to 10 | Days 11 to 12 | Day 12 |
| | | Treatments | Treatments | Treatments | Treatments | Treatments | Treatments | |
| | | A, B, C, D, | A, B, C, D, | A, B, C, D, | A, B, C, D, | A, B, C, D, | A, B, C, D, | |
| | | E or F^{b} | E or F^{b} | E or F^{b} | E or F^{b} | E or F^{b} | E or F^{b} | |

^{a}A minimum 1-day washout seperated each of the six treatments.
^{b}Subjects were randomized to one of six sequences to receive Treatments A, B, C, D, E, F.
Note:
Treatment A: 4.5 g JZP-258 taken with 60 mL water under fasting conditions
Treatment B: 4.5 g Xyrem taken with 60 mL water under fasting conditions
Treatment C: 4.5 g JZP-258 taken with 60 mL water under fed conditions
Treatment D: 4.5 g Xyrem taken with 60 mL water under fed conditions
Treatment E: 4.5 g Xyrem taken with 240 mL water under fasting conditions
Treatment F: 4.5 g JZP-258 taken with 240 mL water under fasting conditions

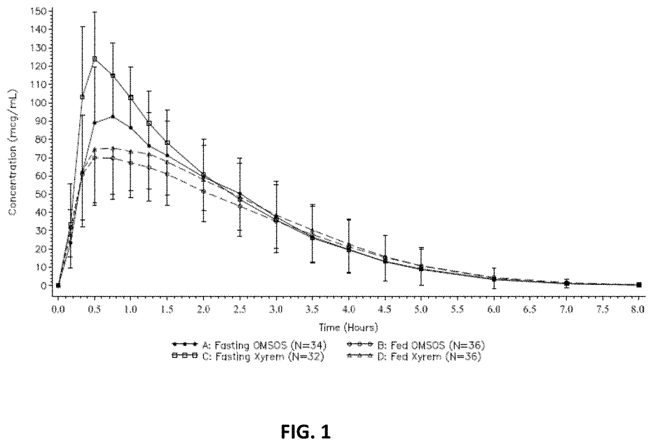

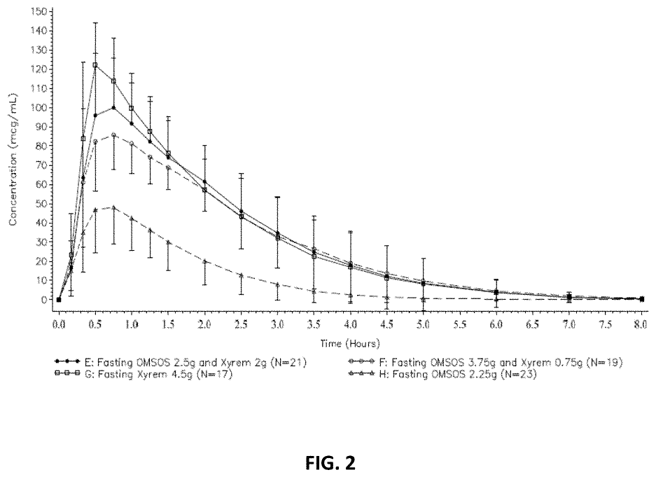

Results
The PK characteristics of JZP-258 were similar to Xyrem (e.g., supra-dose proportionality, and reduced Cmax under fed conditions);
The AUC between Xyrem and JZP-258 were bioequivalent under the same fasted and fed conditions;
However, the Cmax between Xyrem and JZP-258 were not bioequivalent, in that JZP-258 had: a) an approximately 20% lower Cmax compared with Xyrem under fasted conditions, b) a slightly longer time to maximum concentration compared with Xyrem under fasted conditions, and c) a lesser food effect compared with Xyrem. (see FIG. 1, FIG. 2 and data in Table 3)

TABLE 3
Summary of PK Parameters for Part 1 and Part 2 of the Study.
Summary of Results:
Pharmacokinetic:
The mean (CV %) oxybate PK
parameters for Part 1 and Part 2 are presented below:

| | Mean (CV %) | | | | |
|---|---|---|---|---|---|
| | Cₘₐₓ | Tₘₐₓ^{a} | t_{1/2} | AUC [symbol] | AUC [symbol] |
| Treatment | (μg/mL) | (h) | (h) | (μg · h/mL) | (μg · h/mL) |

Part 1 (PK Completer Population, N = 30)

| | | | | | |
|---|---|---|---|---|---|
| Trt A, 4.5 g | 101.8 | 0.75 | 0.57 | 235.4 | 236.5 |
| JZP-258 Fasted | (21.2) | (0.33-1.50) | (21.9) | (32.3) | (32.3) |
| Trt B, 4.5 g | 77.4 | 0.75 | 0.62 | 213.3 | 214.8 |
| JZP-258 Fed | (25.0) | (0.33-2.50) | (27.0) | (33.9) | (34.0) |
| Trt C, 4.5 g | 135.7 | 0.50 | 0.57 | 263.9 | 266.2 |
| Xyrem Fasted | (14.8) | (0.33-1.00) | (28.0) | (30.0) | (30.1) |
| Trt D, 4.5 g | 84.3 | 0.75 | 0.57 | 228.0 | 229.6 |
| Xyrem Fed | (31.3) | (0.33-2.50) | (20.2) | (33.5) | (33.5) |

Part 2 (PK Completer Population, N = 16)

| | | | | | |
|---|---|---|---|---|---|
| Trt E, KZP-258 | 112.3 | 0.64 | 0.73 | 240.0 | 241.4 |
| 2.5 g & Xyrem | (22.2) | (0.33-0.75) | (61.2) | (39.6) | (39.5) |
| 2 g Fasted | | | | | |
| Trt F, KZP-258 | 97.0 | 0.75 | 0.95 | 223.4 | 225.3 |
| 3.65 g & Xyrem | (17.0) | (0.33-2.00) | (65.8) | (33.4) | (33.6) |
| 0.75 g Fasted | | | | | |
| Trt G, Xyrem | 130.9 | 0.50 | 0.73 | 246.6 | 248.2 |
| 4.5 g Fasted | (16.5) | (0.33-0.75) | (28.2) | (36.3) | (36.3) |
| Trt H, JZP-258 | 51.3 | 0.75 | 0.55^{b} | 77.7 | 81.0 |
| 2.25 g Fasted | (40.1) | (0.33-3.00) | (31.5) | (49.4) | (47.5) |

Source: Table 14.2.2-3a and b
^{a}Median (min-max)
^{b}n = 15 for this parameter in this treatment group [symbol]
indicates data missing or illegible when filed

### Example 2

Because bioequivalence for Cmax was not demonstrated for JZP-258, a Phase 3 efficacy and safety study to support registration of JZP-258 was conducted.

Primary Objective: To evaluate the efficacy of JZP-258 in the treatment of cataplexy in subjects with narcolepsy.

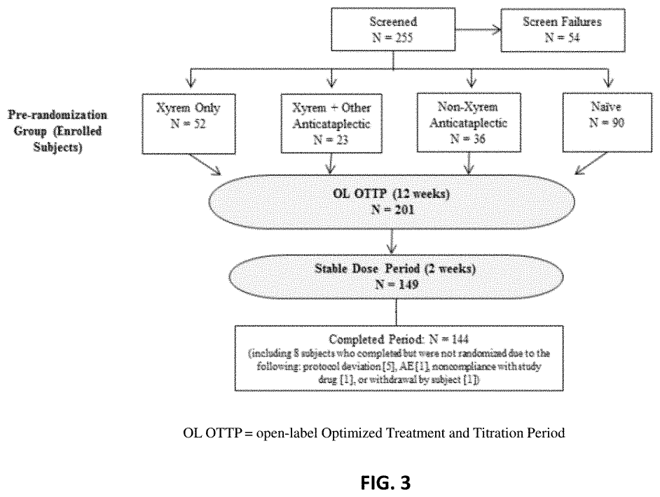

Study Design: This study involved multiple groups of patients with narcolepsy at study entry (FIG. 3), two of which were pretreated with Xyrem and transitioned to JZP-258 as follows:
Patients only treated with Xyrem as an anticataplectic at study entry were switched from Xyrem to JZP-258 (gram for gram) and remained on this JZP-258 dose for a minimum of 2 weeks. If needed for treatment optimization, the dose of JZP-258 was titrated during the subsequent 8 weeks to a stable, tolerable, and effective dose, at the discretion of the investigator
Patients taking Xyrem with other drugs aimed to treat the cataplexy symptom of narcolepsy (“other anticataplectics”) for at least two months prior to screening were switched from Xyrem to JZP-258 (gram for gram) and remained on this JZP-258 dose for a minimum of 2 weeks. Following this 2-week period, subjects were tapered off the additional anticataplectic over a minimum period of 2 weeks and up to 8 weeks. If needed for optimization, the dose of JZP-258 was further titrated to a stable, tolerable, and effective dose during this 8-week period.

Subjects must have been maintained on an unchanged, tolerable, and effective dose of JZP-258 (per the investigator's judgment) alone for at least 2 weeks prior to entering the 2-week Stable Dose Period. During the 2-week Stable Dose Period, subjects remained on the stable JZP-258 dose, unchanged, for 2 weeks. The baseline number of weekly cataplectic attacks and baseline EDS scores, as well as other secondary endpoints (as applicable), were evaluated during this period.

Results:

Provided that JZP-258 was not bioequivalent to Xyrem (see Example 1), it was expected that Xyrem patients would have migrated to a different dose of JZP-258 by the end of the 8-week titration period; however, this was not observed in the study. Unexpectedly, of the subjects who switched from Xyrem to JZP-258 and entered the Stable Dose Period (N=59 overall), the majority (69.5%) remained on the same dose strength (Table 4); for those patients who changed dose, the change was generally within 1.5 grams; i.e., within one incremental dose change.

TABLE 4
Number (%) of Subjects Who Changed Xyrem Total Nightly Dose (gram) at
Study Entry to JZP258 Total Nightly Dose (gram) in Stable Dose Period (OL Stable-Dose Period
Safety Population).

| | Pre-Randomization Group | | |
|---|---|---|---|
| | | Xyrem + | |
| | | Other | |
| | Xyrem Only | Anticataplectic | Total |
| Characteristic | (N = 45) | (N = 14) | (N = 59) |
| number (%) of subjects who increased [a] total | 12 (26.7) | 4 (28.6) | 16 (27.1) |
| nightly dose (gram) | | | |
| change in total nightly dose | 12 | 4 | 16 |
| n | | | |
| Mean (SD) | 1.292 (0.838) | 2.313 (1.375) | 1.547 (1.050) |
| Median | 1.000 | 2.000 | 1.000 |
| Min., Max. | 0.50, 3.00 | 1.00, 4.25 | 0.50, 4.25 |
| number (%) of subjects with change in total | | | |
| nightly dose (gram) | | | |
| 0.5 | 2 (4.4) | 0 | 2 (3.4) |
| 1 | 7 (15.6) | 1 (7.1) | 8 (13.6) |
| 1.5 | 1 (2.2) | 0 | 1 (1.7) |
| 2 | 0 | 2 (14.3) | 2 (3.4) |
| 3 | 2 (4.4) | 0 | 2 (3.4) |
| 4.25 | 0 | 1 (7.1) | 1 (1.7) |
| number (%) of subjects who stay on [a] the same | 31 (68.9) | 10 (71.4) | 41 (69.5) |
| total dose (gram) | | | |
| number (%) of subjects who decreased [a] total | 2 (4.4) | 0 | 2 (3.4) |
| nightly dose (gram) | | | |
| change in total nightly dose | 2 | 0 | 2 |
| n | | | |
| Mean (SD) | −1.250 | | −1.250 |
| | (0.354) | | (0.354) |
| Median | −1.250 | | −1.250 |
| Min., Max. | −1.50, −1.00 | | −1.50, −1.00 |
| number (%) of subjects with change in total nightly | | | |
| dose (gram) | | | |
| −1.5 | 1 (2.2) | 0 | 1 (1.7) |
| −1 | 1 (2.2) | 0 | 1 (1.7) |

As shown in Table 5, for patients who switched from Xyrem® to JZP-258 the median number of dose adjustments required to reach stable total nightly dose was 0 (i.e. no dose adjustment required after switching from Xyrem® to JZP-258 on gram for gram basis) and the median time to reach stable total nightly dose (days) was 1 day.

TABLE 5
Total Nightly Dose During SDP, Time to Reach Stable Total Nightly Dose, and
Number of JZP-258 Dose Adjustments by Treatment at Study Entry (Efficacy Population).

| | | | | |
|---|---|---|---|---|
| SXB | SXB + Other | Other | Anticataplectic | |
| Only | Anticataplectics | Anticataplectics | Naive | Total |
| (n = 41) | (n = 14) | (n = 21) | (n = 58) | (N = 134) |

Total nightly dose (g/night)

| | | | | | |
|---|---|---|---|---|---|
| Mean | 7.59 | 8.29 | 7.41 | 6.90 | 7.33 |
| SD | 1.38 | 1.12 | 1.31 | 1.47 | 1.44 |
| Median | 7.50 | 9.00 | 7.50 | 7.00 | 7.50 |
| Minimum, | 4.5, 9,0 | 6.0, 9.0 | 4.5, 9.0 | 3.0, 9.0 | 3.0, 9.0 |
| maximum | | | | | |

Time to reach stable total nightly dose (days)

| | | | | | |
|---|---|---|---|---|---|
| Mean | 14.5 | 15.7 | 45.5 | 39.0 | 30.1 |
| SD | 21.37 | 24.59 | 18.57 | 20.86 | 24.56 |
| Median | 1.0 | 1.0 | 50.0 | 36.5 | 29.0 |
| Minimum, | 1, 84 | 1, 64 | 5, 73 | 1, 81 | 1, 84 |
| maximum | | | | | |

Number of dose adjustments to reach stable total nightly dose

| | | | | | |
|---|---|---|---|---|---|
| Mean | 1.0 | 0.8 | 3.5 | 2.6 | 2.1 |
| SD | 1.99 | 1.48 | 1.47 | 1.35 | 1.87 |
| Median | 0.0 | 0.0 | 3.0 | 3.0 | 2.0 |
| Minimum, | 0, 8 | 0, 5 | 1, 7 | 0, 6 | 0, 8 |
| maximum | | | | | |

The overall AE profile of JZP-258 was consistent with that previously observed for Xyrem®. Treatment-emergent adverse events (TEAEs) that occurred in >5% of total participants during the open-label optimized treatment and titration period (OLOTTP) by treatment at study entry (safety population) were headache, nausea, dizziness, cataplexy (worsening from baseline), decreased appetite, diarrhea and nasopharyngitis (Table 6).

TABLE 6
TEAEs in ≥5% of Total Participants During OLOTTP by Treatment at Study
Entry (Safety Population)^{a}.

| | Xyrem | Xyrem + Other | Other | Anticataplectic | |
|---|---|---|---|---|---|
| | Only | Anticataplectics | Anticataplecties | Naive | Total |
| TEAEs, n (%) | (n = 52) | (n = 23) | (n = 36) | (n = 90) | (N = 201) |
| Participants with | 30 (57.7) | 19 (82.6) | 30 (83.3) | 70 (77.8) | 149 |
| ≥1 TEAE | | | | | (74.1) |
| Headache | 7 (13.5) | 3 (13.0) | 7 (19.4) | 24 (26.7) | 41 (20.4) |
| Nausea | 2 (3.8) | 1 (4.3) | 6 (16.7) | 14 (15.6) | 23 (11.4) |
| Dizziness | 1 (1.9) | 1 (4.3) | 6 (16.7) | 13 (14.4) | 21 (0.4) |
| Cataplexy^{b} | 0 | 11 (47.8) | 6 (16.7) | 1 (1.1) | 18 (9.0) |
| Decreased appetite | 0 | 1 (4.3) | 2 (5.6) | 12 (13.3) | 15 (7.5) |
| Diarrhea | 4 (7.7) | 0 | 0 | 7 (7.8) | 11 (5.5) |
| Nasopharyngitis | 2 (3.8) | 0 | 3 (8.3) | 5 (5.6) | 10 (5.0) |

OLOTTP, open-label optimized treatment and titration period;
Xyrem ® , sodium oxybate;
TEAE, treatment-emergent adverse event.
^{a}Defined as all participants who took at least 1 dose of study drug.
^{b}Worsening from baseline.

All, documents, patents, patent applications, publications, product descriptions, and protocols which are cited throughout this application are incorporated herein by reference in their entireties for all purposes.

What is claimed:

1. A method for switching a patient who is currently being administered sodium oxybate to a mixed salt oxybate composition, the method comprising:
administering a therapeutically effective amount of a mixed salt oxybate to a patient who has cataplexy or excessive daytime sleepiness with narcolepsy and is being treated with sodium oxybate, wherein the amount of the sodium oxybate and the mixed salt oxybate are within 10% on an oxybate dosing strength basis.

2. The method of claim 1, wherein the mixed salt oxybate comprises sodium oxybate, potassium oxybate, magnesium oxybate and calcium oxybate, and wherein the mixed salt oxybate comprises about 5%-40% sodium oxybate (wt/wt %).

3. The method of claim 2, wherein the mixed salt oxybate comprises about 5%-40% of sodium oxybate (wt/wt %), about 10%-40% of potassium oxybate (wt/wt %), about 5%-30% of magnesium oxybate (wt/wt %), and about 20%-80% of calcium oxybate (wt/wt %).

4. The method of claim 3, wherein the mixed salt oxybate comprises about 8% mol. equiv. of sodium oxybate, about 23% mol. equiv. of potassium oxybate, about 21% mol. equiv. of magnesium oxybate and about 48% mol. equiv. calcium oxybate.

5. The method of any one of claims 1-4, wherein the patient is sensitive to high sodium intake.

6. The method of any one of claims 1-5, wherein the patient is in heart failure.

7. The method of any one of claims 1-5, wherein the patient is hypertensive.

8. The method of any one of claims 1-5, wherein the patient has renal impairment.

9. The method of any one of claims 1-5, wherein the patient is at risk for stroke.

10. The method of any one of claims 1-9, wherein about 0.25 g-10.0 g, 2.0 g-10.0 g; about 3.0 g-9.5 g; or about 4.5 g-9.0 g of the mixed salt oxybate is administered per day.

11. The method of claim 10, wherein the mixed salt oxybate is administered twice per day.

12. The method of claim 10, wherein the mixed salt oxybate is administered once per day.

13. The method of any one of claims 1-12, wherein about 4.5 g of the mixed salt oxybate is administered per day.

14. The method of claim 13, wherein about 2.25 g of the mixed salt oxybate is administered twice per day.

15. The method of any one of claims 1-12, wherein about 6.0 g of the mixed salt oxybate is administered per day.

16. The method of claim 15, wherein about 3.0 g of the mixed salt oxybate is administered twice per day.

17. The method of any one of claims 1-12, wherein about 7.5 g of the mixed salt oxybate is administered per day.

18. The method of claim 17, wherein about 3.75 g of the mixed salt oxybate is administered twice per day.

19. The method of any one of claims 1-12, wherein about 9.0 g of the mixed salt oxybate is administered per day.

20. The method of claim 19, wherein about 4.5 g of the mixed salt oxybate is administered twice per day.

21. The method of any one of claims 1-20, wherein the mixed salt oxybate composition is a liquid.

22. The method of claim 21, wherein the concentration of the mixed salt oxybate in the liquid is from 350 mg/ml-650 mg/ml, or about 450 mg/ml-550 mg/ml.

23. The method of claim 21, wherein the concentration of the mixed salt oxybate in the liquid is about 0.5 g/mL.

24. The method of any one of claims 1-23, wherein the patient is treated for cataplexy.

25. The method of any one of claims 1-23, wherein the patient is treated for excessive daytime sleepiness in patients with narcolepsy.

26. The method of any one of claims 1-25, wherein the mixed salt oxybate is administered at bedtime.

27. The method of any one of claims 1-26 wherein the mixed salt oxybate is administered at bedtime and about 2.5 h-4 h after the bedtime administration.

28. A method for treating cataplexy or excessive daytime sleepiness in patients with narcolepsy, the method comprising
switching a patient who is administered sodium oxybate to a mixed salt oxybate, wherein the switching comprises administering a therapeutically effective amount of the mixed salt oxybate to the patient and wherein the amount of sodium oxybate and mixed salt oxybate are within 5% on an oxybate dosing strength basis.

29. The method of claim 28, wherein the mixed salt oxybate comprises sodium oxybate, potassium oxybate, magnesium oxybate and calcium oxybate, and wherein the mixed salt oxybate comprises about 5%-40% sodium oxybate (wt/wt %).

30. The method of claim 29, wherein the mixed salt oxybate comprises about 5%-40% of sodium oxybate (wt/wt %), about 10%-40% of potassium oxybate (wt/wt %), about 5%-30% of magnesium oxybate (wt/wt %), and about 20%-80% of calcium oxybate (wt/wt %).

31. The method of claim 30, wherein the mixed salt oxybate comprises about 8% mol. equiv. of sodium oxybate, about 23% mol. equiv. of potassium oxybate, about 21% mol. equiv. of magnesium oxybate and about 48% mol. equiv. of calcium oxybate.

32. The method of any one of claims 28-31, wherein the patient is sensitive to high sodium intake.

33. The method of any one of claims 28-32, wherein the patient is in heart failure.

34. The method of any one of claims 28-32, wherein the patient is hypertensive.

35. The method of any one of claims 28-32, wherein the patient has renal impairment.

36. The method of any one of claims 28-32, wherein the patient is at risk for stroke.

37. The method of any one of claims 28-36, wherein about 2.0 g-10.0 g; about 3.0 g-9.5 g; or about 4.5 g-9.0 g of the mixed salt oxybate is administered per day.

38. The method of claim 37, wherein the mixed salt oxybate is administered twice per day.

39. The method of claim 37, wherein the mixed salt oxybate is administered once per day.

40. The method of any one of claims 28-39, wherein about 4.5 g of the mixed salt oxybate is administered per day.

41. The method of claim 40, wherein about 2.25 g of the mixed salt oxybate is administered twice per day.

42. The method of any one of claims 28-39, wherein about 6 g of the mixed salt oxybate is administered per day.

43. The method of claim 42, wherein about 3.0 g of the mixed salt oxybate is administered twice per day.

44. The method of any one of claims 28-39, wherein about 7.5 g of the mixed salt oxybate is administered per day.

45. The method of claim 44, wherein about 3.75 g of the mixed salt oxybate is administered twice per day.

46. The method of any one of claims 28-39, wherein about 9.0 g of the mixed salt oxybate is administered per day.

47. The method of claim 46, wherein about 4.5 g of the mixed salt oxybate is administered twice per day.

48. The method of any one of claims 28-47, wherein the mixed salt oxybate composition is a liquid.

49. The method of claim 48, wherein the concentration of the mixed salt oxybate in the liquid is from 350 mg/ml-650 mg/ml, or about 450 mg/ml-550 mg/ml.

50. The method of claim 48, wherein the concentration of the mixed salt oxybate in the liquid is about 0.5 g/mL.

51. The method of any one of claims 28-50, wherein the patient is treated for cataplexy.

52. The method of any one of claims 28-50, wherein the patient is treated for excessive daytime sleepiness in patients with narcolepsy.

53. The method of any one of claims 28-52, wherein the mixed salt oxybate is administered at bedtime.

54. The method of any one of claims 28-53, wherein the mixed salt oxybate is administered at bedtime and about 2.5 h-4 h after the bedtime administration.

55. A method for substituting a mixed salt oxybate composition for a sodium oxybate composition in a patient treated for cataplexy or excessive daytime sleepiness in patients with narcolepsy, the method comprising:
a. Determining whether a patient treated with a therapeutically effective amount of sodium oxybate is sensitive to high sodium intake; and
b. If the patient is sensitive to high sodium intake, then administering a therapeutically effective amount of a mixed salt oxybate to the patient,
wherein the amount of the sodium oxybate and mixed salt oxybate are the same on an oxybate dosing strength basis.

56. The method of claim 55, wherein the mixed salt oxybate comprises sodium oxybate, potassium oxybate, magnesium oxybate and calcium oxybate, and wherein the mixed salt oxybate comprises about 5%-40% sodium oxybate (wt/wt %).

57. The method of claim 56, wherein the mixed salt oxybate comprises about 5%-40% of sodium oxybate (wt/wt %), about 10%-40% of potassium oxybate (wt/wt %), about 5%-30% of magnesium oxybate (wt/wt %), and about 20%-80% of calcium oxybate (wt/wt %).

58. The method of claim 57, wherein the mixed salt oxybate comprises about 8% mol. equiv. of sodium oxybate, about 23% mol. equiv. of potassium oxybate, about 21% mol. equiv. of magnesium oxybate and about 48% mol. equiv. of calcium oxybate.

59. The method of any one of claims 55-58, wherein the patient is in heart failure.

60. The method of any one of claims 55-58, wherein the patient is hypertensive.

61. The method of any one of claims 55-58, wherein the patient has renal impairment.

62. The method of any one of claims 55-58, wherein the patient is at risk for stroke.

63. The method of any one of claims 55-62, wherein about 2.0 g-10.0 g; about 3.0 g-9.5 g; or about 4.5 g-9.0 g of the mixed salt oxybate is administered per day.

64. The method of claim 63, wherein the mixed salt oxybate is administered twice per day.

65. The method of claim 63, wherein the mixed salt oxybate is administered once per day.

66. The method of any one of claims 55-65, wherein about 4.5 g of the mixed salt oxybate is administered per day.

67. The method of claim 66, wherein about 2.25 g of the mixed salt oxybate is administered twice per day.

68. The method of any one of claims 55-65, wherein about 6.0 g of the mixed salt oxybate is administered per day.

69. The method of claim 68, wherein about 3 g of the mixed salt oxybate is administered twice per day.

70. The method of any one of claims 55-65, wherein about 7.5 g of the mixed salt oxybate is administered per day.

71. The method of claim 70, wherein about 3.75 g of the mixed salt oxybate is administered twice per day.

72. The method of any one of claims 55-65, wherein about 9.0 g of the mixed salt oxybate is administered per day.

73. The method of claim 72, wherein about 4.5 g of the mixed salt oxybate is administered twice per day.

74. The method of any one of claims 55-73, wherein the mixed salt oxybate composition is a liquid.

75. The method of claim 74, wherein the concentration of the mixed salt oxybate in the liquid is from 350 mg/ml-650 mg/ml, or about 450 mg/ml-550 mg/ml.

76. The method of claim 74, wherein the concentration of the mixed salt oxybate in the liquid is about 0.5 g/mL.

77. The method of any one of claims 55-75 wherein the patient is treated for cataplexy.

78. The method of any one of claims 55-75, wherein the patient is treated for excessive daytime sleepiness in patients with narcolepsy.

79. The method of any one of claims 55-78, wherein the mixed salt oxybate is administered at bedtime.

80. The method of any one of claims 55-79, wherein the mixed salt oxybate is administered at bedtime and about 2.5 h-4 h after the bedtime administration.

81. The method of any one of claims 1-27, wherein the amount of the sodium oxybate and the mixed salt oxybate are the same on a gram for gram basis.

82. The method of any one of claims 28-54, wherein the switching comprises administering a therapeutically effective amount of the mixed salt oxybate to the patient and wherein the amount of sodium oxybate and mixed salt oxybate are the same on a gram for gram basis.
